(19) United States
(12) Patent Application Publication
(10) Pub. No.: US 2019/0151524 A1
(43) Pub. Date: May 23, 2019

(54) ORGAN RESTRAINT COVERINGS AND COATINGS FOR ATRIAL FIBRILLATION PREVENTION

(71) Applicant: Edwards Lifesciences Corporation, Irvine, CA (US)

(72) Inventors: Stanton J. Rowe, Newport Coast, CA (US); Robert S. Schwartz, Inver Grove Heights, MN (US)

(21) Appl. No.: 16/178,379

(22) Filed: Nov. 1, 2018

Related U.S. Application Data
(60) Provisional application No. 62/587,995, filed on Nov. 17, 2017.

(51) Int. Cl. A61M 1/12; A61M 1/10; A61F 2/24

(52) U.S. Cl. CPC A61M 1/122; A61M 1/125; A61F 7/10; A61F 2/2481; A61M 1/1008

(57) ABSTRACT

A method of restraining expansion of an atrium of a heart involves accessing a heart of a patient, applying a coating over at least a portion of a surface of an atrium of the heart, and at least partially curing the coating to increase the rigidity thereof. Atrial fibrillation prevention.

### RELATED APPLICATION

This application claims priority to U.S. Provisional Application No. 62/587,995, filed Nov. 17, 2017, and entitled ORGAN RESTRAINT COVERINGS AND COATINGS FOR ATRIAL FIBRILLATION PREVENTION, the disclosure of which is hereby incorporated by reference in its entirety.

### BACKGROUND

### Field

The present disclosure generally relates to the field of vascular surgery, such as cardiac surgery.

### Description of Related Art

Patients of cardiac surgery and other vascular operations can develop atrial fibrillation post-operatively due to various conditions and/or factors. Atrial fibrillation is associated with certain health complications, including increased patient mortality, and therefore prevention and/or treatment of atrial fibrillation during surgery and/or post-operatively can improve patient health.

### SUMMARY

In some implementations, the present disclosure relates to a method of restraining expansion of an atrium of a heart. The method comprises accessing a heart of a patient, applying a coating over at least a portion of a surface of an atrium of the heart, and at least partially curing the coating to increase rigidity thereof.

The coating may comprise bio-resorbable material. In certain embodiments, applying the coating and at least partially curing the coating at least partially limit stretching of the atrium. In certain embodiments, applying the coating and at least partially curing the coating at least partially increase elasticity associated with a wall of the atrium. In certain embodiments, applying the coating and at least partially curing the coating at least partially decrease elasticity associated with a wall of the atrium. In certain embodiments, applying the coating comprises brushing the coating onto the surface of the atrium. In certain embodiments, applying the coating comprises spraying the coating onto the surface of the atrium. In certain embodiments, applying the coating comprises expelling the coating from an applicator tip of a syringe.

The coating may have adhesive properties. In certain embodiments, the coating comprises collagen. In certain embodiments, the coating comprises hydrophobic polymer. In certain embodiments, the coating comprises polymer doped with carbon nanotubes. In certain embodiments, the coating comprises oxidized dextran.

In certain embodiments, at least partially curing the coating comprises exposing the coating to light. For example, the light may be ultraviolet (UV) light. The coating may be configured to change color as it cures to provide a visual indication of curing. In certain embodiments, the coating has a Young's modulus of elasticity of between 0.2 MPa and 1.0 MPa when cured. The coating may be configured such that, when cured, a surface of the coating does not adhere to biological tissue coming in contact therewith.

In some implementations, the present disclosure relates to a method of restraining expansion of an atrium of a heart. The method comprises accessing a heart of a patient, and disposing a biocompatible covering over at least a portion of a surface of an atrium of the heart. The biocompatible covering is configured to at least partially restrain outward expansion of the surface of the atrium.

The biocompatible covering may advantageously be bio-resorbable. In certain embodiments, the biocompatible covering comprises a mesh patch. The biocompatible covering may have a Young's modulus of elasticity of between 0.2 MPa and 1.0 MPa. In certain embodiments, the method further comprises trimming the biocompatible covering to fit the surface of the atrium disposing the biocompatible covering. The method may further comprise suturing the biocompatible covering to the heart. In certain embodiments, the method further comprises applying adhesive to one or more of the surface of the atrium and the biocompatible covering, and adhering the biocompatible covering to the surface of the atrium using the adhesive.

In some implementations, the present disclosure relates to an atrial restraint covering comprising a form of biocompatible material shaped to cover a surface of an atrium of a heart, wherein the form of biocompatible material is configured to be secured to the surface of the atrium and at least partially restrict outward expansion thereof.

The form of biocompatible material may be bio-resorbable. In certain embodiments, the form of biocompatible material comprises a mesh patch. In certain embodiments, the form of biocompatible material has a Young's modulus of elasticity of between 0.2 MPa and 1.0 MPa. In certain embodiments, the form of biocompatible material comprises adhesive to adhering to the surface of the atrium.

### BRIEF DESCRIPTION OF THE DRAWINGS

Various embodiments are depicted in the accompanying drawings for illustrative purposes, and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 provides an example cross-sectional view of a human heart.

FIG. 2 illustrates an example cross-sectional representation of a heart experiencing atrial fibrillation.

FIGS. 3 and 4 show anterior and posterior views, respectively, of a human heart.

FIGS. 5 and 6 illustrates anterior and posterior views, respectively, of a heart having an atrial restraint coating or covering applied to one or more atria thereof in accordance with one or more embodiments.

### DETAILED DESCRIPTION

The headings provided herein are for convenience only and do not necessarily affect the scope or meaning of the claimed invention.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Terminology

Certain standard anatomical terms of location are used herein to refer to the anatomy of animals, and namely humans, with respect to the preferred embodiments. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. Spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

Furthermore, references may be made herein to certain anatomical planes, such as the sagittal plane, or median plane, or longitudinal plane, referring to a plane parallel to the sagittal suture, and/or other sagittal planes (i.e., parasagittal planes) parallel thereto. In addition, “frontal plane,” or “coronal plane,” may refer to an X-Y plane that is perpendicular to the ground when standing, which divides the body into back and front, or posterior and anterior, portions. Furthermore, a “transverse plane,” or “cross-sectional plane,” or horizontal plane, may refer to an X-Z plane that is parallel to the ground when standing, that divides the body in upper and lower portions, such as superior and inferior. A “longitudinal plane” may refer to any plane perpendicular to the transverse plane. Furthermore, various axes may be described, such as a longitudinal axis, which may refer to an axis that is directed towards head of a human in the cranial direction and/or directed towards inferior of a human in caudal direction. A left-right or horizontal axis, which may refer to an axis that is directed towards the left-hand side and/or right-hand side of a patient. An anteroposterior axis which may refer to an axis that is directed towards the belly of a human in the anterior direction and/or directed towards the back of a human in the posterior direction.

### Overview

In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.). The contraction of the various heart muscles may be prompted by signals generated by the electrical system of the heart, which is discussed in detail below.

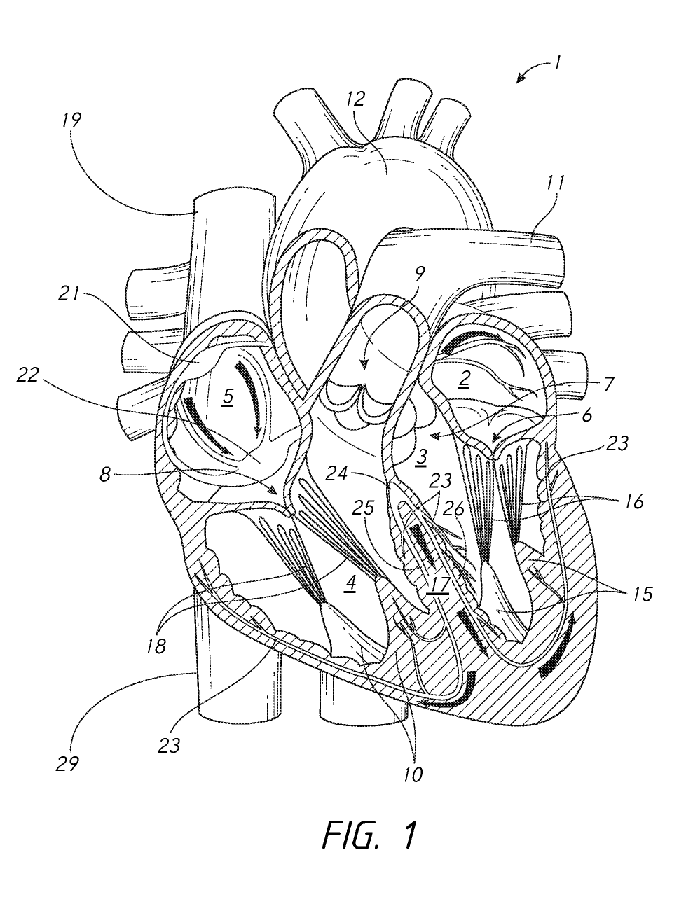

FIG. 1 illustrates an example representation of a heart 1 having various features relevant to certain embodiments of the present inventive disclosure. The heart 1 includes four chambers, namely the left atrium 2, the left ventricle 3, the right ventricle 4, and the right atrium 5. A wall of muscle 17, referred to as the septum, separates the left 2 and right 5 atria and the left 3 and right 4 ventricles. The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (i.e., systole) and open during ventricular expansion (i.e., diastole). The valves of the heart 1 further include the pulmonary valve 9, which separates the right ventricle 4 from the pulmonary artery 11, and may be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

Heart valves may generally comprise a relatively dense fibrous ring, referred to herein as the annulus, as well as a plurality of leaflets or cusps attached to the annulus. Generally, the size and position of the leaflets or cusps may be such that when the heart contracts, the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage.

The atrioventricular (i.e., mitral and tricuspid) heart valves may further comprise a collection of chordae tendineae (16, 18) and papillary muscles (10, 15) for securing the leaflets of the respective valves to promote and/or facilitate proper coaptation of the valve leaflets and prevent prolapse thereof. The papillary muscles (10, 15), for example, may generally comprise finger-like projections from the ventricle wall. With respect to the mitral valve 6, a normal mitral valve may comprise two leaflets (anterior and posterior) and two corresponding papillary muscles 15. When the left ventricle 3 contracts, the intraventricular pressure forces the valve to close, while the chordae tendineae 16 keep the leaflets coapting together and prevent the valve from opening in the wrong direction, thereby preventing blood to flow back to the left atrium 2. With respect to the tricuspid valve 8, the normal tricuspid valve may comprise three leaflets (two shown in FIG. 1) and three corresponding papillary muscles 10 (two shown in FIG. 1). The leaflets of the tricuspid valve may be referred to as the anterior, posterior and septal leaflets, respectively. The valve leaflets are connected to the papillary muscles by the chordae tendineae 18, which are disposed in the right ventricle 4 along with the papillary muscles 10. The right ventricular papillary muscles 10 originate in the right ventricle wall, and attach to the anterior, posterior and septal leaflets of the tricuspid valve, respectively, via the chordae tendineae 18.

### Cardiac Electrical System

The electrical system of the heart generally controls the events associated with the pumping of blood by the heart. With further reference to FIG. 1, the heart 1 comprises different types of cells, namely cardiac muscle cells (also known as cardiomyocytes or myocardiocytes) and cardiac pacemaker cells. For example, the atria (2, 5) and ventricles (3, 4) comprise cardiomyocytes, which are the muscle cells that make up the cardiac muscle. The cardiac muscle cells are generally configured to shorten and lengthen their fibers and provide desirable elasticity to allow for stretching. Each myocardial cell contains myofibrils, which are specialized organelles consisting of long chains of sarcomeres, the fundamental contractile units of muscle cells.

The electrical system of the heart utilizes the cardiac pacemaker cells, which are generally configured to carry electrical impulses that drive the beating of the heart 1. The cardiac pacemaker cells serve to generate and send out electrical impulses, and to transfer electrical impulses cell-to-cell along electrical conduction paths. The cardiac pacemaker cells further may also receive and respond to electrical impulses from the brain. The cells of the heart are connected by cellular bridges, which comprise relatively porous junctions called intercalated discs that form junctions between the cells. The cellular bridges permit sodium, potassium and calcium to easily diffuse from cell-to-cell, allowing for depolarization and repolarization in the myocardium such that the heart muscle can act as a single coordinated unit.

The electrical system of the heart comprises the sinoatrial (SA) node 21, which is located in the right atrium 5 of the heart 1, the atrioventricular (AV) node 22, which is located on the interatrial septum in proximity to the tricuspid valve 8, and the His-Purkinje system 23, which is located along the walls of the left 3 and right 4 ventricles.

A heartbeat represents a single cycle in which the heart's chambers relax and contract to pump blood. As described above, this cycle includes the opening and closing of the inlet and outlet valves of the right and left ventricles of the heart. Each beat of the heart is generally set in motion by an electrical signal generated and propagated by the heart's electrical system. In a normal, healthy heart, each beat begins with a signal from the SA node 21. This signal is generated as the vena cavae (19, 29) fill the right atrium 5 with blood, and spreads across the cells of the right 5 and left 2 atria. The flow of electrical signals is represented by the illustrated shaded arrows in FIG. 1. The electrical signal from the SA node 21 causes the atria to contract, which pushes blood through the open mitral 6 and tricuspid 8 valves from the atria into the left 3 and right 4 ventricles, respectively.

The electrical signal arrives at the AV node 22 near the ventricles, where it may slow for an instant to allow the right 4 and left 3 ventricles to fill with blood. The signal is then released and moves along a pathway called the bundle of His 24, which is located in the walls of the ventricles. From the bundle of His 24, the signal fibers divide into left 26 and right 25 bundle branches through the Purkinje fibers 23. These fibers connect directly to the cells in the walls of the left 3 and right 4 ventricles. The electrical signal spreads across the cells of the ventricle walls, causing both ventricles to contract. Generally, the left ventricle may contract an instant before the right ventricle. Contraction of the right ventricle 4 pushes blood through the pulmonary valve 9 to the lungs (not shown), while contraction of the left ventricle 3 pushes blood through the aortic valve 6 to the rest of the body. As the electrical signal passes, the walls of the ventricles relax and await the next signal.

### Atrial Fibrillation

FIG. 1, as described above, illustrates a normal electrical flow, resulting in a regular heart rhythm that may be associated with a generally healthy heart. However, in certain patients or individuals, various conditions and/or events can result in compromised electrical flow, causing the development and/or occurrence of an abnormal heart rhythm. For example, atrial fibrillation is a condition associated with abnormal electrical flow and/or heart rhythm characterized by relatively rapid and irregular beating of the atria.

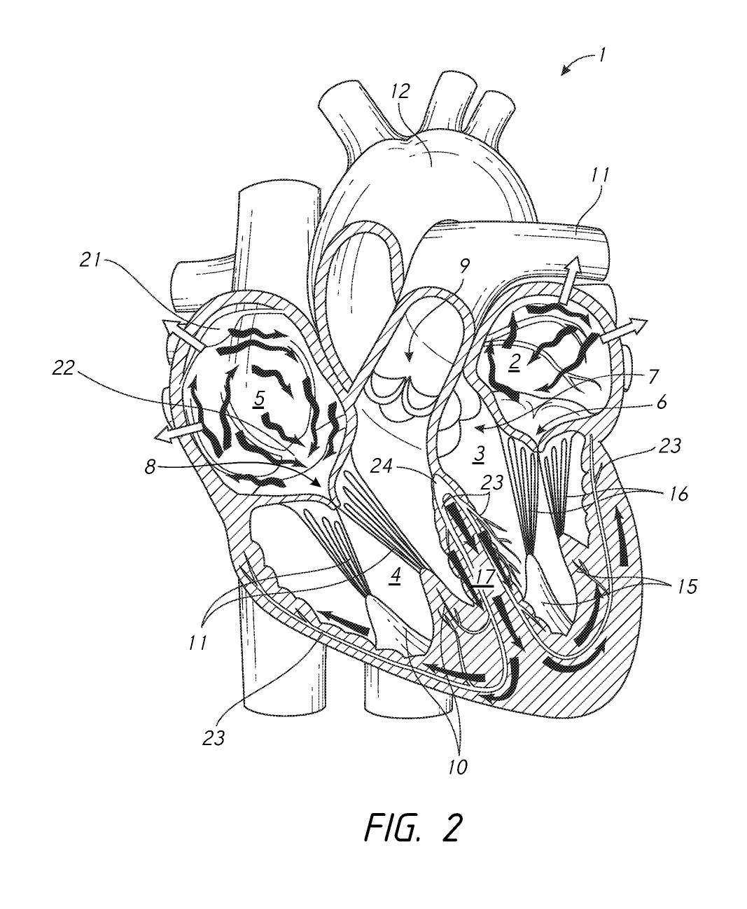

FIG. 2 illustrates an example cross-sectional representation of the heart 1 of FIG. 1 experiencing atrial fibrillation. When atrial fibrillation occurs, the normal regular electrical impulses generated by the sinoatrial (SA) node 21 in the right atrium 5 may become overwhelmed by disorganized electrical impulses, which may lead to irregular conduction of ventricular impulses that generate the heartbeat. The illustrated shaded arrows represent the erratic electrical impulses that can be associated with atrial fibrillation. Atrial fibrillation generally originates in the right atrium 5, that where conduction path disturbances begin.

Various pathologic developments can lead to, or be associated with, atrial fibrillation. For example, progressive fibrosis of the atria may contribute at least in part to atrial fibrillation. The formation of fibrous tissue associated with fibrosis can disrupt or otherwise affect the electrical pathways of the cardiac electrical system due to interstitial expansion associated with tissue fibrosis. In addition to fibrosis in the muscle mass of the atria, fibrosis may also occur in the sinoatrial node 21 and/or atrioventricular node 22, which may lead to atrial fibrillation.

Fibrosis of the atria may be due to atrial dilation, or stretch, in some cases. Dilation of the atria can be due to a rise in the pressure within the heart, which may be caused by fluid overload, or may be due to a structural abnormality in the heart, such as valvular heart disease (e.g., mitral stenosis, mitral regurgitation, tricuspid regurgitation), hypertension, congestive heart failure, or other condition. Dilation of the atria can lead to the activation of the renin aldosterone angiotensin system (RAAS), and subsequent increase in matrix metalloproteinases and disintegrin, which can lead to atrial remodeling and fibrosis and/or loss of atrial muscle mass.

In addition to atrial dilation, inflammation in the heart can cause fibrosis of the atria. For example, inflammation may be due to injury associated with a cardiac surgery, such as a valve repair operation, or the like. Alternatively, inflammation may be caused by sarcoidosis, autoimmune disorders, or other condition. Other cardiovascular factors that may be associated with the development of atrial fibrillation include high blood pressure, coronary artery disease, mitral stenosis (e.g., due to rheumatic heart disease or mitral valve prolapse), mitral regurgitation, hypertrophic cardiomyopathy (HCM), pericarditis, and congenital heart disease. Additionally, lung diseases (such as pneumonia, lung cancer, pulmonary embolism, and sarcoidosis) may contribute to the development of atrial fibrillation in some patients.

### Development of Post-Operative Atrial Fibrillation

In addition to the various physiological conditions described above that may contribute to atrial fibrillation, in some situations, atrial fibrillation may be developed in connection with a vascular operation, such post-operatively in the days following a vascular operation. Various factors may bear on the likelihood of a patient developing post-operative atrial fibrillation, such as age, medical history (e.g., history of atrial fibrillation, chronic obstructive pulmonary disease (COPD)), concurrent valve surgery, withdrawal of post-operative treatment (e.g., beta-adrenergic blocking agents (i.e., beta blocker), angiotensin converting enzyme inhibitors (ACE inhibitor)), beta-blocker treatment (e.g., pre-operative and/or post-operative), ACE inhibitor treatment (e.g., pre-operative and/or post-operative), and/or other factors. Generally, for patients that experience post-operative atrial fibrillation, the onset of atrial fibrillation may occur approximately 2-3 days after surgery.

Atrial dilation/stretching may be considered a primary variable associated with post-operative atrial fibrillation. In some situations, occurrence of post-operative atrial fibrillation may follow, at least in part, the following progression: First, the patient undergoes a surgical procedure, such as a vascular surgical operation (e.g., cardiac surgery). In connection with the operation, the patient may be subject to drug and/or fluid management. For example, the patient may receive post-surgery intravenous (IV) fluid loading and/or diuretic/drug volume management. Such treatment may result in fluid overload, which may lead to atrial stretching due to increased pressure in one or more atria. Atrial stretching may occur over a 1-2-day period, or longer, resulting in dilation of one or both of the atria. Fibrotic atrial tissue may form in connection with atrial stretching. Atrial stretching and/or fibrotic atrial tissue formation may result in an increased incidence of post-operative atrial fibrillation (e.g., 30-40% increased incidence of post-operative atrial fibrillation). In addition, inflammation associated with surgical operations can contribute the onset of post-operative atrial fibrillation, and reduced inflammation may generally correlate to a reduced risk of atrial fibrillation.

Post-operative atrial fibrillation is generally associated with increased patient morbidity, as well as economic burden. For example, post-operative atrial fibrillation is generally associated with increased incidence of congestive heart failure, increased hemodynamic instability, increase renal insufficiency, increased repeat hospitalizations, increased risk of stroke, and increase in hospital mortality and 6-month mortality. Post-operative atrial fibrillation also represents a systemic burden, wherein intensive care unit (ICU) stay, hospital length of stay, hospital charges, and rates of discharge to extended care facilities are increased as a result of post-operative atrial fibrillation.

Furthermore, because an initial incidence of atrial fibrillation generally results in recurring, progressively more severe, episodes of atrial fibrillation in a patient, the consequences of allowing atrial fibrillation to develop post-operatively can be considered particularly severe for a given patient. For example, a given patient may initially experience intermittent/sporadic episodes of atrial fibrillation as a result of post-operative atrial dilation and/or inflammation, with recurring episodes progressively increasing in frequency and/or severity.

### Prevention of Post-Operative Atrial Stretch and Inflammation

The development of atrial fibrillation post-operatively can have a serious negative impact on patient quality of life. As discussed above, atrial stretch and inflammation may represent root causes of post-operative atrial fibrillation in some situations. Therefore, by reducing or restricting atrial stretch and/or inflammation during vascular surgery, or over a period of time thereafter, incidences of post-operative atrial fibrillation can be reduced. The majority of post-operative atrial fibrillation instances may occur within the first two days after surgery, and therefore, prevention of post-operative atrial stretch and/or inflammation may be particularly significant during the initial days after surgery.

Generally, atrial diameter expansion of greater than 5 mm may be correlated with chronic atrial fibrillation in some cases. Furthermore, increase in atrial circumference of greater than 10%, and/or increase in atrial volume of greater than 8.5 mL may be associated with chronic atrial fibrillation. Therefore, embodiments disclosed herein may be designed to limit or restrict atrial stretch to prevent expansion of atrial diameter by 5 mm or more, increase in circumferential stretch by greater than 10%, and/or increase in atrial volume by 8.5 mL or more in order to reduce incidences of atrial fibrillation. With regard to fluid overload, in some situations, the introduction of around 1.5 additional liters of fluid to a patient's vascular system may be correlated with increased rates of atrial fibrillation. Generally, the greater the amount of fluid added, the greater the amount of atrial stress that may be experienced by the patient.

In some implementations, the present disclosure provides a means for restricting atrial stretching in either or both of the left and right atria, and/or the reduction of inflammation associated with the atria, for a post-operative period after a surgical procedure, thereby reducing the likelihood of onset of post-operative atrial fibrillation. For example, embodiments disclosed herein may be suitable for restricting atrial stretching and/or reducing inflammation for a period of up to five days after a surgical procedure. In some implementations, a post-operative atrial fibrillation prevention device may be implanted or applied at the time of surgery, but may advantageously be removed at a later time. For example, in some embodiments, an atrial fibrillation prevention device may be removed at or about the time that chest drainage tubes associated with a surgical operation are removed, which may correspond with a time period approximately five days after completion of the surgery, or other time period.

### Atrial Restraint Coatings and Coverings

As described in detail above, fluid volume overload in the vascular system of a patient, and in particular within the atria, can cause an increase in atrial pressure. When exposed to elevated atrial pressures, atrial tissue may be inclined to stretch over time. Various mechanisms, coatings, coverings, devices, and processes are disclosed herein for at least partially restraining the left and/or right atrium from stretching to thereby reduce the risk of post-operative atrial fibrillation. Atrial restraint devices and methods disclosed herein may advantageously at least partially restrict the expansion or stretching of atrial tissue, while allowing for desirable expansion of the atria in order to accommodate the proper contraction and expansion of the atria typically associated with each heartbeat cycle. For example, that diameter of an atrium may change by approximately 2 mm per beat for a healthy heart. Therefore, in some implementations, coatings/coverings and methods for restraining atrial stretch according to the present disclosure may advantageously accommodate approximately 2 mm per beat of diameter change of the atria, but at least partially limit stretching beyond that.

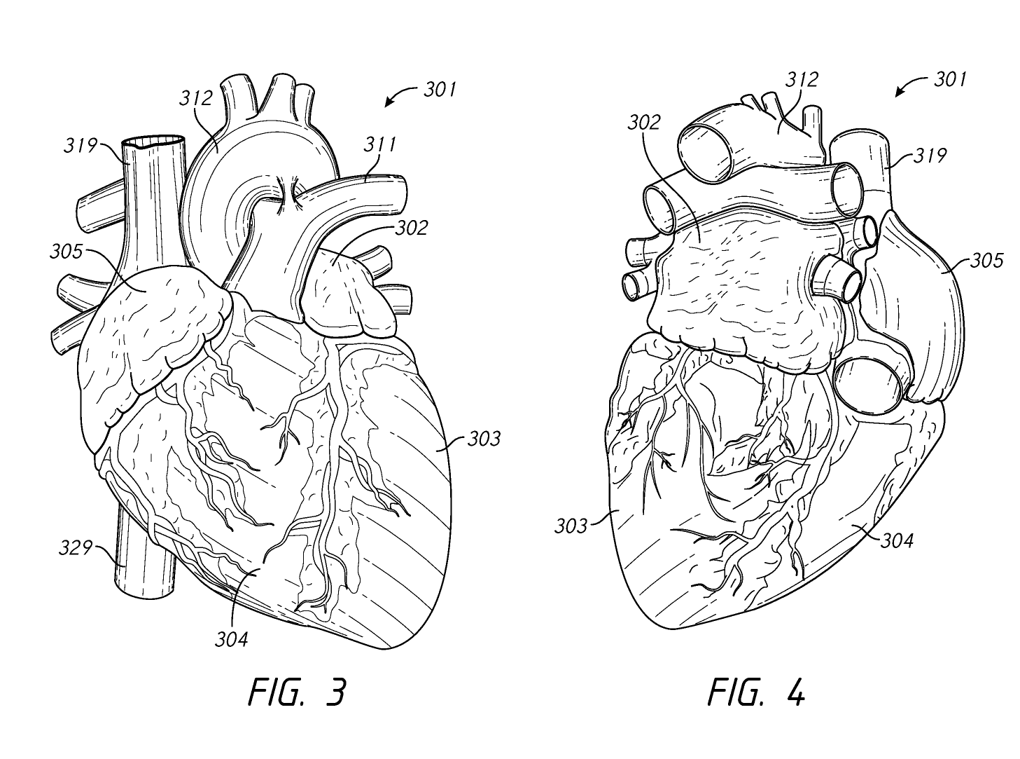

As described above, the outward expansion of stretching of the right and/or left atrium of the heart can result in development of atrial fibrillation in some patients. For reference, FIGS. 3 and 4 show anterior and posterior views, respectively, of a heart 301 showing the surfaces of the right atrium 305 and the left atrium 302. Certain embodiments disclosed herein provide coatings and/or coverings, and methods associated therewith, for restraining the outward expansion of the atrial walls, which may be prone to stretching and expansion due to increased fluid pressure therein, which may be caused by fluid overload and/or other fluid management conditions.

Stretching or expansion of the atria may be restrained and/or prevented at least in part through the application of external pressure on the outside surface of the atria. Some embodiments disclosed herein relate to the placement of at least partially rigid materials, structures, or forms on the surface of the atria to thereby restrain expansion thereof. For example, in some embodiments, atrial restraint is achieved through the operative application of biocompatible adhesive coating onto the atrial surface(s), which may serve to at least partially stiffen the atrial tissue, thus preventing or limiting atrial stretching. As described in detail above, the reduction of atrial stretching may reduce the incidence of postoperative atrial fibrillation.

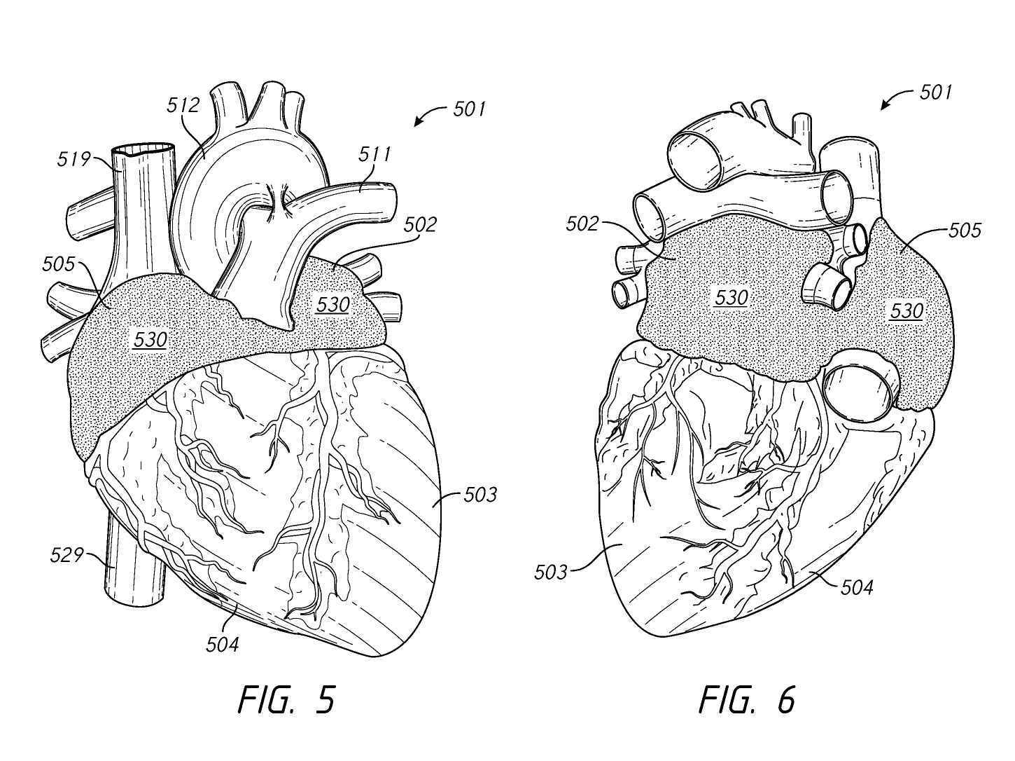

FIGS. 5 and 6 illustrates anterior and posterior views, respectively, of a heart 501 having atrial restraint form or agent 530 applied to one or more atria thereof, to thereby restrain the expansion and/or stretching of the atria. As shown, the right atrium 505 and left atrium 502 have applied thereto a restraining coating or covering 530. Although certain embodiments are disclosed herein in the context of biocompatible adhesive coatings, it should be understood that such embodiments may incorporate other types of materials, such as patches, casts, and/or other forms comprising any suitable or desirable at least partially rigid material, or material that may become rigid through application of a certain material, process, or treatment.

The application of, for example, a biocompatible adhesive coating may at least partially prevent atrial stretching by way of thickening of the atrial wall. Furthermore, atrial restraint coatings and/or coverings as disclosed herein may further serve to at least partially increase the atrial walls' modulus of elasticity.

In some embodiments, the coating or covering 530 shown in FIGS. 5 and 6 may comprise a bio-resorbable spray-on coating. Such coating may have certain properties that may promote the restraint characteristics of the coating and/or facilitate the application thereof to the atrial wall. For example, the coating may comprise hydrophobic polymer, which may be configured to adhere to the atrial wall tissue. For example, such polymer may be doped with carbon nanotubes (e.g., nanoscale pillars), and/or oxidized dextran, which may provide improved adhesion characteristics.

In certain embodiments, the coating or covering 530 may comprise light-cured adhesive material, which may become cured in the presence of ultraviolet (UV), or other wavelengths of light. In some embodiments, the covering or coating 530 comprises a light-cured coating/adhesive that is configured to change color when cured so as to inform the physician or technician of the coverage areas of the cured adhesive. That is, the color of the cured coating may provide a visual indication that the coating is cured in a particular area. In light-cured adhesive embodiments, the coating may be configured to cure within 20 minutes or less, such as within a few minutes. The surface of the cured adhesive may be such as to prevent adhesion of the treated atria to the chest cavity. The atrial restraint material may advantageously have relatively high-viscosity adhesive properties, which may be preferable to promote and/or facilitate control of the application of the material to the target area of the atrium or atria.

In some embodiments, the coating or covering 530 comprises an at least partially flexible or elastic material, which may allow for some degree of stretching to promote the proper contraction of the atria in connection with heartbeat cycles. For example, the coating or covering 530 may have a Young's modulus of elasticity (E) between 0.2-1.0 MPa.

In some embodiments, the coating or covering 530 comprises bio-resorbable material that is configured or designed to degrade over a period of time after application thereof. For example, the coating may degrade over a period of between 1 to 6 months, or over period of time. The coating may comprise collagen, or the like.

With respect to embodiments in which the atria are restrained using biocompatible adhesive material, such coating may be applied in any suitable or desirable manner. For example, in some embodiments, the coating material may be applied using a brushed-on application, wherein a brush or similar type of tool or device is utilized to apply and/or spread the material over the surface of the atrium. In some implementations, the restraint material may be sprayed onto the atrial surface, using some type of spray application nozzle or tool. In some implementations, the atrial restraint material may be applied using a sponge-on application, wherein a sponge-type tool may be used to spread and/or apply the material onto the atrial surface. In some implementations, the atrial restraint material may be applied using a syringe having an applicator tip, wherein restraint material may be expelled from the applicator tip over the target area of the atrium or atria. In some implementations, the atrial restraint material (e.g. adhesive polymer) may be percutaneously injected via a catheter to one or more additional treatment locations, such as the left atrial appendage, cerebral aneurysm, or the like, and may be cured via a light source that may be incorporated into the delivery catheter.

In certain embodiments, the covering or coating 530 shown in FIGS. 5 and 6 may comprise a tape or sheet form, which may be pre-cut or cut in real-time to fit the desired target surface area of the atrium or atria. That is, rather than applying an amorphous coating to the atrial surface, embodiments disclosed here may provide use of a shaped form of biocompatible material that may be placed over the atrial surface. For example, one or more portions of the covering 530 may comprise a patch that may be placed over the target area. The patch may comprise any suitable material and may advantageously have a suitable degree of rigidity to restrain the stretching of the atrial tissue once the patch is placed and/or secured. Such patch may comprise any suitable or desirable material, such as mesh, cloth, or the like. Such a patch or covering may be secured to the atria in any suitable or desirable way, such as through suturing, or through the use of adhesives or other attachment tool or mechanism. The covering 530 is configured to at least partially restrain outward expansion of the surfaces of the atria. With respect to embodiments of patch-type coverings 530, such coverings may be secured to the surface of the atria using adhesive. For example, the covering 530 may have adhesive properties, and/or adhesive may be applied to the covering 530 and/or surface of the atria in order to adhere the covering 530 to the atrial surface.

In some implementations, atrial restraint may be achieved through the use of synthetic and/or memory metal (e.g. Nitinol) mesh. For example, restraint mesh may comprise a restraint patch, such as a Silastic patch. Such patch may be trimmed or customized to fit a particular patient's atria or atrium. In some embodiments, an atrial restraint patch may be sutured in place over the atrium. Atrial restraint patches may advantageously comprise bio-resorbable material, such that the patch need not be removed from the patient after its useful life. In some embodiments, restraint is achieved through the use of polymer film, which may be deposited or applied to the regions of the atria that are desired to be restrained. However, such films may not provide desirably uniform restraint force in some implementations. Use of synthetic mesh may advantageously provide desirable restraint, and may be formed to fit a desired shape atria. Mesh restraint patches may be trimmed or cut using scissors or other tools, such that a surgeon may be able to fit or trim the patch him or herself at the time of an operation. Furthermore, restraint patches in accordance with embodiments of the present disclosure may comprise any suitable or desirable material, including rigid or non-rigid cloths or forms. Such patches/forms may be fixed to the atrial surface, or other biological tissue or surface, in any suitable or desirable manner

### Additional Embodiments

Depending on the embodiment, certain acts, events, or functions of any of the processes described herein can be performed in a different sequence, may be added, merged, or left out altogether. Thus, in certain embodiments, not all described acts or events are necessary for the practice of the processes. Moreover, in certain embodiments, acts or events may be performed concurrently.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

What is claimed is:

1. A method of restraining expansion of an atrium of a heart, the method comprising:
accessing a heart of a patient;
applying a coating over at least a portion of a surface of an atrium of the heart; and
at least partially curing the coating to increase rigidity thereof.

2. The method of claim 1, wherein the coating comprises bio-resorbable material.

3. The method of claim 1, wherein said applying the coating and said at least partially curing the coating at least partially limit stretching of the atrium.

4. The method of claim 1, wherein said applying the coating and said at least partially curing the coating at least partially increase elasticity associated with a wall of the atrium.

5. The method of claim 1, wherein said applying the coating comprises brushing the coating onto the surface of the atrium.

6. The method of claim 1, wherein said applying the coating comprises spraying the coating onto the surface of the atrium.

7. The method of claim 1, wherein said applying the coating comprises expelling the coating from an applicator tip of a syringe.

8. The method of claim 1, wherein the coating has adhesive properties.

9. The method of claim 1, wherein the coating comprises collagen.

10. The method of claim 1, wherein the coating comprises hydrophobic polymer.

11. The method of claim 1, wherein the coating comprises polymer doped with carbon nanotubes.

12. The method of claim 1, wherein the coating comprises oxidized dextran.

13. The method of claim 1, wherein said at least partially curing the coating comprises exposing the coating to light.

14. The method of claim 13, wherein the light is ultraviolet (UV) light.

15. The method of claim 1, wherein the coating is configured to change color as it cures to provide a visual indication of curing.

16. The method of claim 1, wherein the coating has a Young's modulus of elasticity of between 0.2 MPa and 1.0 MPa when cured.

17. The method of claim 1, wherein the coating is configured such that, when cured, a surface of the coating does not adhere to biological tissue coming in contact therewith.

18. A method of restraining expansion of an atrium of a heart, the method comprising:
accessing a heart of a patient; and
disposing a biocompatible covering over at least a portion of a surface of an atrium of the heart;
wherein the biocompatible covering is configured to at least partially restrain outward expansion of the surface of the atrium.

19. The method of claim 18, wherein the biocompatible covering is bio-resorbable.

20. The method of claim 18, wherein the biocompatible covering comprises a mesh patch.

21. The method of claim 18, wherein the biocompatible covering has a Young's modulus of elasticity of between 0.2 MPa and 1.0 MPa.

22. The method of claim 18, further comprising trimming the biocompatible covering to fit the surface of the atrium after said disposing the biocompatible covering.

23. The method of claim 18, further comprising suturing the biocompatible covering to the heart.

24. The method of claim 18, further comprising:
applying adhesive to one or more of the surface of the atrium and the biocompatible covering; and
adhering the biocompatible covering to the surface of the atrium using the adhesive.

25. An atrial restraint covering comprising:
a form of biocompatible material shaped to cover a surface of an atrium of a heart;
wherein the form of biocompatible material is configured to be secured to the surface of the atrium and at least partially restrict outward expansion thereof.

26. The atrial restraint covering of claim 25, wherein the form of biocompatible material is bio-resorbable.

27. The atrial restraint covering of claim 25, wherein the form of biocompatible material comprises a mesh patch.

28. The atrial restraint covering of claim 25, wherein the form of biocompatible material has a Young's modulus of elasticity of between 0.2 MPa and 1.0 MPa.

29. The atrial restraint covering of claim 25, wherein the form of biocompatible material comprises adhesive to adhering to the surface of the atrium.
